(19) United States
(12) Patent Application Publication
(10) Pub. No.: US 2017/0119461 A1
(43) Pub. Date: May 4, 2017

(54) Electrosurgical Apparatus with Temperature Sensing and Methods of use thereof

(71) Applicant: Kyphon SÁRL, Neuchâtel (CH)

(72) Inventors: Neil Godara, Milton (CA); Robert Harrison, Milton (CA); Kathryn Atwell, Etobicoke (CA); Natalia Burachynsky, Toronto (CA); Michael Same, Toronto (CA)

(21) Appl. No.: 15/333,310

(22) Filed: Oct. 25, 2016

Related U.S. Application Data
(60) Provisional application No. 62/248,019, filed on Oct. 29, 2015.

(51) Int. Cl. A61B 18/14; A61B 18/12

(52) U.S. Cl. CPC A61B 18/1477; A61B 18/148; A61B 18/1206; A61B 2018/00577

(57) ABSTRACT

Embodiments of apparatus comprise a thermocouple sensor positioned within an introducer capable of being placed in either soft or bony tissues, and a generator system for monitoring the thermocouple and providing the temperature information to a user and/or the generator system for use in controlling energy delivery. An embodiment of a method includes positioning a thermocouple sensor within an introducer capable of being placed in either soft or bony tissues, a generator system monitoring the thermocouple, and providing the temperature information to a user and/or the generator system which use the temperature information for controlling energy delivery.

### RELATED APPLICATIONS

This application claims priority to U.S. Provisional Patent Application No. 62/248,019 filed Oct. 29, 2015; all of which is incorporated by reference herein in its entirety.

### BACKGROUND

The present invention relates to medical devices used to deliver energy to tissue. More specifically, the present invention relates to energy delivery medical devices having temperature sensors.

The variability of tumors poses a challenge for ablation, especially within bone tissue, since current tumor ablation systems for use in bone tissue are unable to adequately monitor temperatures at a distance (fixed or variable) from the ablation electrode. This limitation is particularly significant in cases where a tumor targeted for ablation is located close to a tissue that should be preserved, for example, a sensitive neural structure that could be inadvertently damaged during tumor ablation. It would be advantageous if a tumor ablation system included temperature monitoring and an ablation probe that cooperates with an electrosurgical generator to provide comprehensive treatment information and facilitate dynamic adjustment of energy delivery during treatment.

### SUMMARY

The present invention contemplates an ablation system for delivering energy to a targeted area of living tissue and for monitoring a temperature of a proximate area of living tissue near the targeted area of living tissue to prevent damage to the proximate area of living tissue. The ablation system includes an ablation probe introducer having an outer surface and an inner surface, a proximal end having a proximal wall, and an opposite distal end having a distal wall, the distal wall having a distal aperture, and the ablation probe introducer having a central longitudinal axis passing through the proximal wall and the distal wall of the ablation probe introducer. The ablation system also includes an ablation probe positioned within the ablation probe introducer, the ablation probe including a plurality of ablation probe electrodes at a distal end of the ablation probe to delivery energy to the targeted area of living tissue. The ablation system further includes a temperature sensor proximate to the ablation probe, the temperature sensor determining the temperature of the proximate area of living tissue. The ablation system additionally includes a power generator that supplies power to the ablation probe and the temperature sensor, the ablation probe and the temperature sensor being electrically coupled to the power generator, the power generator adapted to receive temperature information from the temperature sensor and adapted to adjust the power supplied to the ablation probe to prevent damage to the area of living tissue near the targeted area of living tissue.

The present invention also contemplates a method of delivering energy by an ablation system to a targeted area of living tissue and for monitoring a temperature of a proximate area of living tissue near the targeted area of living tissue to prevent damage to the proximate area of living tissue. The method includes utilizing an ablation probe introducer having an outer surface and an inner surface, a proximal end having a proximal wall, and an opposite distal end having a distal wall, the distal wall of the ablation probe introducer having a distal aperture, and the ablation probe introducer having a central longitudinal axis passing through the proximal wall and the distal wall of the ablation probe introducer. The method also includes inserting an ablation probe into the ablation probe introducer, the ablation probe including a plurality of ablation probe electrodes at a distal end of the ablation probe to deliver energy to the targeted area of living tissue. The method further includes utilizing a temperature sensor proximate to the ablation probe, the temperature sensor determining the temperature of the proximate area of living tissue. The method additionally includes utilizing a power generator that supplies power to the ablation probe and the temperature sensor, the ablation probe and the temperature sensor being electrically coupled to the power generator. The method also includes receiving, at the power generator, temperature information from the temperature sensor. The method further includes adjusting, at the power generator, the power supplied to the ablation probe to prevent damage to the area of living tissue near the targeted area of living tissue.

The present invention also contemplates the ablation system where the ablation probe introducer includes an ablation probe introducer hub, the targeted area of living tissue is a tumor, the power generator is an electrosurgical generator, the electrosurgical generator automatically adjusts the power supplied to the ablation probe depending on the temperature information from the temperature sensor, the electrosurgical generator adjusts the power supplied to the ablation probe by user input, the ablation system includes a thermocouple probe that is separate from the ablation probe introducer, the ablation system also includes a thermocouple probe introducer including an outer surface and an inner surface, a proximal end having a proximal wall, and an opposite distal end having a distal wall, the distal wall of the thermocouple probe introducer having a distal aperture, the thermocouple probe introducer having a central longitudinal axis of the thermocouple probe introducer, and the thermocouple probe positioned within the thermocouple probe introducer, the temperature sensor is positioned at the distal end of the ablation probe, and the ablation system also includes a cooling unit connected to the ablation probe for providing cooling fluid to the ablation probe.

The present invention further contemplates the method that includes connecting an ablation probe introducer hub to the ablation probe introducer, inserting the ablation probe into a tumor to deliver energy to the tumor, the utilizing of the power generator is utilizing an electrosurgical generator that supplies the power to the ablation probe and the temperature sensor, the adjusting of the power supplied to the ablation probe is automatically adjusted depending on the temperature information received from the temperature sensor, the adjusting of the power supplied to the ablation probe is adjusted by user input, utilizing a thermocouple probe electronically coupled to the power generator, the thermocouple probe providing additional temperature information to the power generator, inserting the thermocouple probe into the ablation probe introducer, utilizing a thermocouple probe introducer including an outer surface and an inner surface, a proximal end having a proximal wall, and an opposite distal end having a distal wall, the distal wall of the thermocouple probe introducer having a distal aperture, the thermocouple probe introducer having a central longitudinal axis of the thermocouple probe introducer, and the thermocouple probe positioned within the thermocouple probe introducer, and utilizing a cooling unit connected to the ablation probe for providing cooling fluid to the ablation probe.

### BRIEF DESCRIPTION OF THE DRAWINGS

In order that the invention may be readily understood, embodiments of the invention are illustrated by way of examples in the accompanying drawings, in which:

FIG. 1 is an illustration of a system in accordance with an embodiment of the present invention;

FIG. 2 is an illustration of a system in accordance with an alternative embodiment of the present invention;

FIG. 3 is an illustration of a vertebral body indicating some of the locations a thermocouple probe could be used to measure tissue temperature;

FIG. 4 is a diagram of an alternative embodiment of the present invention which includes a connector hub;

FIG. 5 is an illustration of an embodiment of an alternative ablation probe introducer; and

FIG. 6 is an illustration of an example of track burning.

### DETAILED DESCRIPTION

Described herein are examples of embodiments of the present invention that include a thermocouple sensor probe positioned within an introducer capable of being placed in either soft or bony tissue, and a generator system that monitors the thermocouple probe to provide information to the user and/or the generator system for altering the energy delivery profile as required by the patient presentation.

In one broad aspect, embodiments of the present invention comprise a thermocouple sensor positioned within an introducer capable of being placed in either soft or bony tissues, and a generator system that monitors the thermocouple and provides the temperature information to a user and/or the generator system for use in controlling energy delivery.

Features of this broad aspect include the temperature information being used directly by the generator to (a) control energy delivery, (b) halt treatment, or (c) provide an alert to the user. In alternative embodiments, the information is displayed onscreen so that the user can make decisions about prolonging treatment, halting treatment, or altering the treatment.

As a feature of this aspect, systems disclosed herein have been used for bony tumor ablation, with or without soft tissue interaction.

In a further broad aspect, embodiments of the present invention comprise a system including (1) a first device for energy delivery and temperature sensing (e.g., an ablation probe with a thermocouple); (2) a second device for temperature sensing (e.g., a thermocouple probe); and (3) an electrosurgical generator that monitors the temperature sensors of the first and second devices simultaneously and displays the temperature data, whereby the user may alter the energy delivery profile based on the temperature data. In alternative embodiments of the system, an algorithm of the electrical generator adjusts energy delivery based on the temperature data.

With specific reference now to the drawings, it is stressed that the particulars shown are by way of example and for purposes of illustrative discussion of certain embodiments of the present invention only. Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

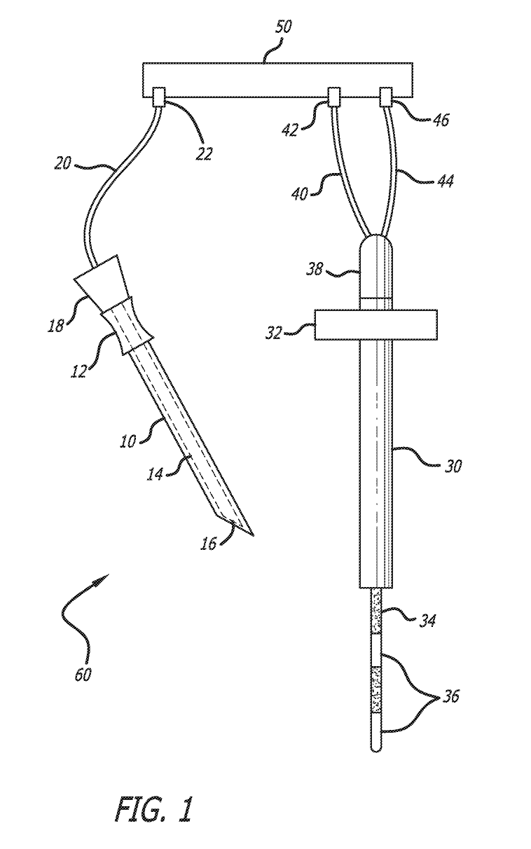

System 60 of FIG. 1 includes a thermocouple probe introducer 10, a thermocouple probe 14, an ablation probe introducer 30, an ablation probe 34, and a electrosurgical generator 50. Thermocouple probe introducer 10 can be placed in soft or bony tissue as required, and has a temperature sensor (thermocouple probe 14) contained within. In typical use, thermocouple probe 14 is inserted into thermocouple probe introducer 10 until thermocouple probe handle 18 abuts thermocouple probe introducer hub 12.

Making further reference to the example of FIG. 1, ablation probe 34 is inserted into ablation probe introducer 30 with ablation probe electrodes 36 exposed beyond the distal end of the introducer. In typical embodiments, ablation probe 34 is inserted into ablation probe introducer 30 until ablation probe handle 38 contacts ablation probe introducer hub 32. In some embodiments, ablation probe handle 38 and ablation probe introducer hub 32 lock together. The temperature sensor of ablation probe 34 communicates with electrosurgical generator 50 via ablation probe sensor cable 40 which is connected to electrosurgical generator 50 by ablation probe sensor cable connector 42. Electrosurgical generator 50 supplies power to ablation probe 34 through power cable 44 which is connected to electrosurgical generator 50 by power cable connector 46. When in use, ablation probe electrodes 36 are normally positioned inside a tumor (not shown in drawing) that is targeted for ablative energy delivery. Ablation probe 34 includes a temperature sensor, typically a thermocouple, which can be considered the primary temperature sensor (or primary thermocouple) since it provides a temperature close to, or in, the tissue to which energy is delivered during ablation. In some embodiments, the ablation probe temperature sensor is located at the distal tip of ablation probe 34. Further details about temperature sensors (thermocouples) of the probes are found in the applications cited herein below. Thermocouple probe 14 can be considered the secondary temperature sensor (or secondary thermocouple).

In most embodiments, electrosurgical generator 50 is a radiofrequency (RF) generator system capable of monitoring thermocouple probe 14 and delivering RF electrical energy to ablation probe 34. In typical embodiments of system 60, electrosurgical generator 50 is capable of delivering RF electrical energy whilst using signals from both the thermocouple probe 14 and the temperature sensor of ablation probe 34 to alter the profile of the electrical energy delivered to the targeted tumor.

In the embodiment of FIG. 1, thermocouple probe cable 20 is attached to thermocouple probe handle 18 and to electrosurgical generator 50 (via thermocouple probe connector 22) to enable communication between the probe and generator. In most embodiments, the generator displays temperature information of both the thermocouple probe 14 and RF ablation probe's temperature sensor, and thereby provides the user feedback regarding the temperatures in and near the ablation zone. Optionally, electrosurgical generator 50 can halt or modify energy delivery based on the feedback provided by the secondary thermocouple (thermocouple probe 14). For example, if the secondary thermocouple is placed near body tissue that should not be ablated, and a temperature measured using the secondary thermocouple is high enough to damage the non-target tissue, the generator can be stopped from delivering energy.

In the embodiment of FIG. 1, thermocouple probe 14 is positioned away from ablation probe electrodes 36 such that thermocouple probe 14 supplies a temperature reading of tissue that is spaced apart from the center of the ablative volume (the location of ablation probe electrodes 36). In some cases, thermocouple probe 14 is used to obtain the temperature of bone tissue, while in other cases it is used to obtain the temperature of soft tissue. In the embodiments of FIG. 1, thermocouple probe tip 16 is be positioned off to the side of ablation probe 34, i.e., laterally. In alternative embodiments, thermocouple probe tip 16 is positioned in-line with ablation probe 34. It is thereby possible for system 60 to be used in bone tissue (or soft tissue) for monitoring temperatures at a variable distance from ablation probe electrodes 36, and to use the temperature information to control energy delivery from the tumor ablation probe.

In some embodiments, the generator system simultaneously monitors thermocouple probe 14 and the temperature sensor of ablation probe 34, and provides information regarding the temperatures in and near the ablation zone to the user and/or an algorithm of the generator system whereby the energy delivery profile is altered if required. For example, if the user positions the secondary thermocouple near a sensitive vascular structure and a temperature reading from that location indicates that energy delivery could damage the vascular structure, energy delivery can be stopped. In some particular embodiments, the algorithm of electrosurgical generator 50 accepts the distance between thermocouple probe 14 and ablation probe 34 when it is entered by the user into electrosurgical generator 50, and the generator uses the distance and the temperature information from ablation probe 34 and thermocouple probe 14 to alter the energy delivery profile so as to provide safety and maximize the efficiency and effectiveness of the ablation procedure. In other embodiments, the distance between the thermocouples is communicated to the generator by navigation and/or imaging systems that can determine the distances directly. In yet other embodiments, the distance is approximated by the generator using tissue characteristics such as impedance.

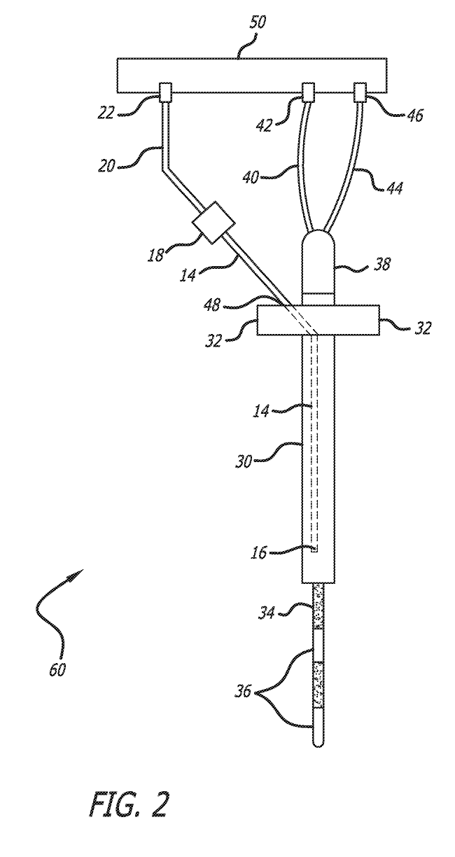

FIG. 2 is an illustration of an alternative embodiment of system 60 which does not include a separate thermocouple probe introducer 10. Instead, thermocouple probe 14 is inserted through opening 48 of ablation probe introducer 30 and into a lumen of the introducer. The inserted portion of thermocouple probe 14 is shown in broken line in FIG. 2. In some embodiments, thermocouple probe 14 is inserted into the same lumen that contains ablation probe 34, while in other embodiments, thermocouple probe 14 is inserted into a separate lumen. Thermocouple probe 14 can be advanced or partially withdrawn within the lumen to vary the distance of thermocouple probe tip 16 from ablation probe electrodes 36.

In the embodiments of FIGS. 1 and 2, thermocouple probe cable 20, ablation probe sensor cable 40, and power cable 44, are connected directly to electrosurgical generator 50. Some alternative embodiments of electrosurgical generator 50 include an additional thermocouple probe connector 22 such that an additional thermocouple probe 14 can be connected to the generator, and some further embodiments include two or more additional thermocouple probe connectors.

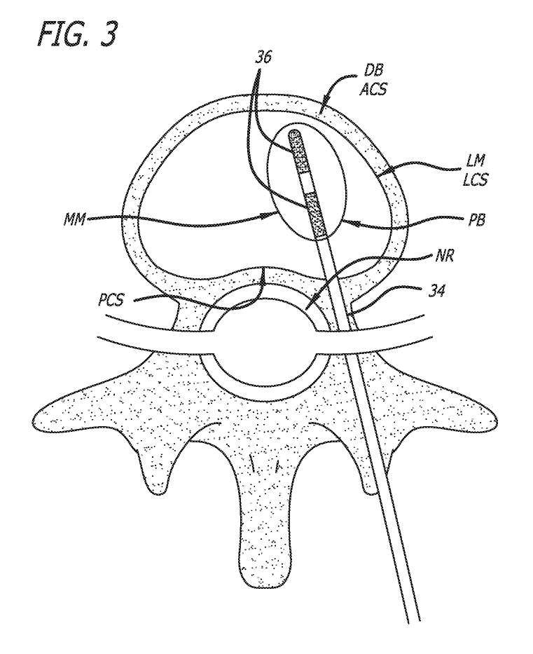

FIG. 3 is an illustration of a vertebral body indicating some of the locations (positions of interest) thermocouple probe 14 (not shown in drawing) can be used to measure tissue temperature. FIG. 3 illustrates an ablation probe positioned in a vertebral body with ablation probe electrodes 36 surrounded by a lesion. Positions of interest are marked in the drawing. To ensure effectiveness, temperature readings can be measured surrounding the lesion at the lesion's distal boundary (DB), proximal boundary (PB), medial margin (MM), and lateral margin (LM). For reasons of safety, temperature readings can be measured at the posterior cortical shell (PCS), the anterior cortical shell (ACS), the lateral cortical shell (LCS), and the nerve root (NR).

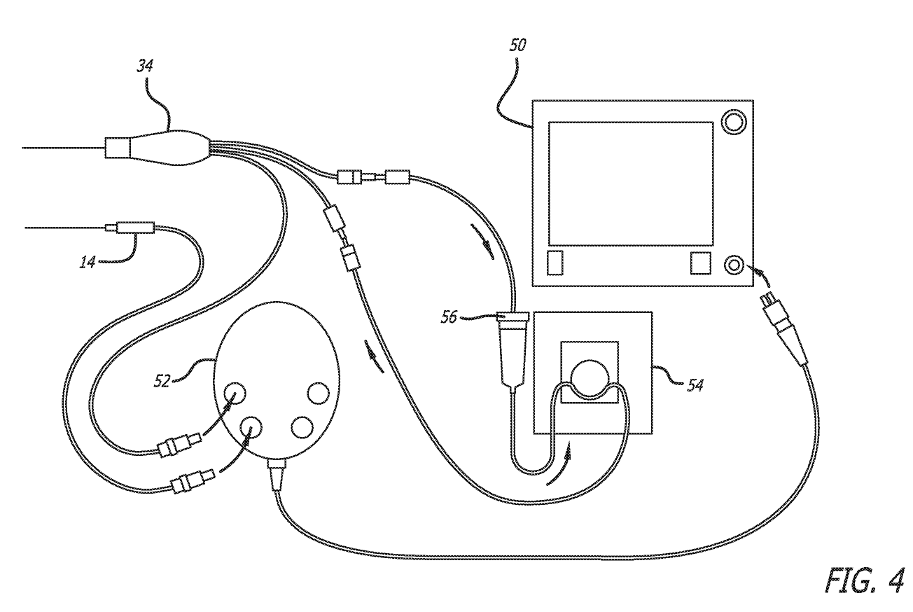

FIG. 4 is a diagram of an alternative embodiment of the system which includes a connector hub 52. In the embodiment of FIG. 4, the ablation probe sensor cable and the power cable are contained in the same cable casing, and the thermocouple probe cable, the ablation probe sensor cable, and power cable are all connected to the connector hub 52. A hub-to-generator cable connects connector hub 52 to electrosurgical generator 50. Connector hub 52 facilitates handling and identification of the probes. The connector hub 52 illustrated in FIG. 4 includes a second ablation probe connector for connecting a second ablation probe 34 and a second thermocouple probe connector for connecting a second thermocouple probe 14. Some alternative embodiments of the connector hub include additional thermocouple probe connectors.

The embodiment of FIG. 4 also includes a pump unit 54 and a tubing kit 56 connecting the pump unit 54 to ablation probe 34 for providing cooling fluid to ablation probe 34, which in typical embodiments is internally cooled. Further details about the internal cooling of probes are found in the applications cited herein below.

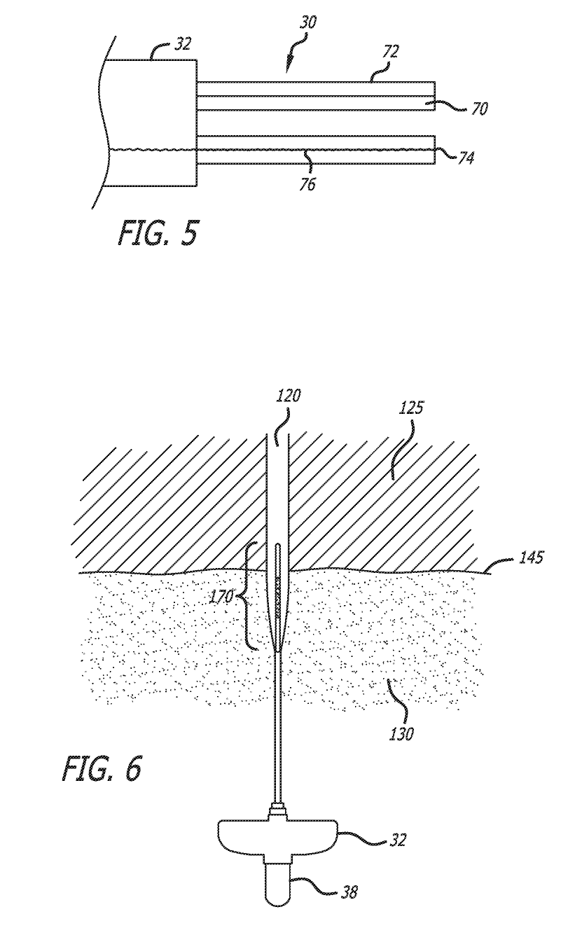

FIG. 5 is an illustration of an embodiment of an ablation probe introducer 30 having an incorporated secondary thermocouple. Ablation probe introducer 30 is comprised of two tubes, inner tube 70 and outer tube 72, which are coaxially aligned. In some embodiments there is a space between inner tube 70 and outer tube 72 (not shown in drawing). The secondary thermocouple is comprised of thermocouple wires 76 that extend from ablation probe introducer hub 32, between inner tube 70 and outer tube 72, to the distal end of the introducer. Typically, the distal ends of thermocouple wires 76 are welded together to form thermocouple junction 74. Thermocouple junction 74 may be welded to one or both of inner tube 70 and outer tube 72. In some embodiments of the apparatus, ablation probe handle 38 and ablation probe introducer hub 32 lock together whereby the distance between the temperature sensor of the ablation probe and the secondary thermocouple is fixed.

As previously disclosed, some embodiments of electrosurgical generator 50 include an algorithm for controlling energy delivery. In some such embodiments, the algorithm has two modes of operation: an ablation mode and a safety mode. The mode of operation is selected by the user based on the positioning of thermocouple probe 14 (in particular, the positioning of thermocouple probe tip 16). For example, in typical embodiments, if thermocouple probe tip 16 is positioned at the lesion's desired distal boundary, or inside the desired lesion, the ablation mode is selected, and, in typical embodiments, if thermocouple probe tip 16 is positioned at a location of safety concern, such as a nerve root, the safety mode is selected. In some embodiments the user makes a selection from a selection menu, while in other embodiments the user inputs a selection using a touch screen interface. Further embodiments include the user inputting a selection using other input methods known to those skilled in the art. When in ablation mode, the algorithm delivers energy to the treatment site until such time that the thermocouple probe tip measures a temperature indicative of having surpassed an ablation threshold. When in safety mode, the algorithm provides safe energy delivery by altering energy delivery prior to temperatures being reached at the thermocouple probe tip that could be damaging to tissue, which, in typical embodiments, includes stopping energy delivery if necessary.

In typical embodiments, the secondary thermocouple sensor is adapted for bony applications, i.e., it can be used in or around bony tissue. In some embodiments, the user can input whether the secondary thermocouple is in hard tissue (bone), close to hard tissue, or in soft tissue, and the algorithm adjusts or matches to the tissue condition for providing effective tissue ablation and safety. The apparatus and methods disclosed herein can be used within the bones that make up the vertebral column (at the discretion of a physician) as well as within other bones of a patient (i.e. not just in a vertebral body as shown in the example of FIG. 3).

Some embodiments of the algorithm/apparatus further include a retract mode used for track burning. Track burning includes withdrawing a probe through a tissue and at least partially concurrently delivering energy from the probe to heat a layer of tissue surrounding the probe to a temperature sufficient for thermal coagulation necrosis of cells. An example of track burning is illustrated in FIG. 6 (FIG. 6 of U.S. Publication No. US20140257265, the application of which is herein incorporated-by-reference in its entirety), wherein an active tip 170 of a probe extends through an introducer assembly. The introducer and probe have been withdrawn together, and delivery of energy through active tip 170 has formed a coagulated tissue path 120 through hard tissue 125, tissue boundary 145, and soft tissue 130. Depending on the configuration of the apparatus, ablation probe handle 38 and ablation probe introducer hub 32 may be locked together or fittingly engaged to facilitate controlled withdrawal of the apparatus. In some embodiments (e.g. FIG. 1), active tip 170 is spaced apart from the distal tip of the introducer. It is typical in track burning for a probe's active tip to extend beyond an introducer, whereby the apparatus is withdrawn together while the probe tip delivers energy to coagulate tissue surrounding the path of withdrawal.

Some embodiments of the retract mode used for track burning comprise using the ablation probe temperature sensor without the secondary thermocouple. Examples of such embodiments are described in U.S. application Ser. No. 14/195,972, entitled “SYSTEMS AND METHODS FOR TRACK COAGULATION.”

Some embodiments of the retract mode comprise using the secondary thermocouple (or secondary temperature sensor) without the ablation probe temperature sensor. Referring to the example of FIG. 5, a thermocouple junction 74 at the distal tip of introducer can be used for temperature monitoring for track burning. In embodiments of the apparatus wherein active tip 170 is spaced apart from the distal tip of the introducer, thermocouple junction 74 is proximal of the active electrodes and consequently in a region of tissue having a lower temperature than the tissue closer to (and surrounding) the electrodes. When the tissue adjacent thermocouple junction 74 is heated sufficiently to result in some degree of coagulation, the higher temperature around the electrodes results in a relatively greater degree of coagulation, which typically produces effective track burning.

Another embodiment of the retract mode including use of the secondary thermocouple (or secondary temperature sensor) without the ablation probe temperature sensor uses apparatus having the general configuration of the example of FIG. 2. In FIG. 2, ablation probe electrodes 36 are distal of the tip of ablation probe introducer 30. In some embodiments of ablation probe introducer 30, the lumen containing thermocouple probe 14 extends to the distal tip of the introducer whereby the thermocouple probe can be extended beyond the distal end of introducer to contact tissue. This configuration enables the thermocouple probe 14 to measure tissue temperature during track burning. In some embodiments, the tissue temperature is measured adjacent the tip of the introducer, and in other embodiments the thermocouple probe is further advanced to measure a temperature of tissue closer (or adjacent) to the electrodes. Since such embodiments of the method include thermocouple probe 14 being close to or touching ablation probe 34, some embodiments of thermocouple probe 14 comprise a thin outer layer of electrical insulation to keep electrical energy delivered by ablation probe 34 from flowing into thermocouple probe 14. Such a layer of insulation is typically thin enough that it does not function as a thermal insulation, whereby, as understood by one skilled in the art, the layer of insulation allows heat to flow through it, and thermocouple probe 14 to accurately monitor tissue temperature.

Some embodiments of the retract mode comprise using both the secondary thermocouple (or secondary temperature sensor) and the ablation probe temperature sensor. Such embodiments include using a secondary thermocouple positioned as described in the above examples, while simultaneously measuring temperature with an ablation probe temperature sensor, and an algorithm analyzing the data from both temperature sensors. In some embodiments the algorithm controls the energy delivery such that tissue surrounding the electrodes has a sufficient temperature for track ablation while limiting energy delivery if the tissue temperature is in excess of the temperature needed for coagulation by a significant (possibly defined) limit, thereby reducing or avoiding the ablation of excess tissue.

In all of the above embodiments of track burning the ablation probe temperature sensor and/or the secondary thermocouple can be used to determine the rate of retraction and to facilitate the safety and efficacy of track burning. In embodiments which comprise using both the secondary thermocouple and the ablation probe temperature sensor, the rate of temperature change between the two temperature sensors can be utilized. For example, when each temperature sensor passes by a certain point in the tissue, the absolute temperature value measured by each sensor can be monitored, and the temperature difference the two temperature sensors determined.

Some of the examples of the above embodiments of track burning include withdrawing the probe via a path that traverses at least some bone tissue. Some of the examples of the above embodiments of track burning include delivering energy in a bipolar manner. U.S. Publication No. US20140257265, published Sep. 11, 2014, discloses further details of track burning, such as an algorithm controlling generator functions. The details of track burning disclosed in said application may be included in all of the above described embodiments of retract mode.

Further details about probes used for ablation and the use thereof are found in U.S. application Ser. No. 13/643,310, entitled “ELECTROSURGICAL DEVICE AND METHODS,” PCT filing date of Apr. 15, 2011, U.S. application Ser. No. 13/660,353, entitled “ELECTROSURGICAL DEVICE AND METHODS”, filed Oct. 25, 2012, U.S. application Ser. No. 14/195,972, entitled “SYSTEMS AND METHODS FOR TRACK COAGULATION,” filed Mar. 4, 2014, and PCT Application No. PCT/182014/059846, entitled “ELECTROSURGICAL MAPPING TOOLS AND METHODS,” filed 14 Mar. 2014, all of which are herein incorporated by reference in their entirety into the specification.

The embodiment(s) of the invention described above are intended to be exemplary only. The scope of the invention is therefore intended to be limited solely by the scope of the appended claims.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. An ablation system for delivering energy to a targeted area of living tissue and for monitoring a temperature of a proximate area of living tissue near the targeted area of living tissue to prevent damage to the proximate area of living tissue, the ablation system comprising:
an ablation probe introducer having an outer surface and an inner surface, a proximal end having a proximal wall, and an opposite distal end having a distal wall, the distal wall having a distal aperture, and the ablation probe introducer having a central longitudinal axis passing through the proximal wall and the distal wall of the ablation probe introducer; an ablation probe positioned within the ablation probe introducer, the ablation probe including a plurality of ablation probe electrodes at a distal end of the ablation probe to delivery energy to the targeted area of living tissue; a temperature sensor proximate to the ablation probe, the temperature sensor determining the temperature of the proximate area of living tissue; and
a power generator that supplies power to the ablation probe and the temperature sensor, the ablation probe and the temperature sensor being electrically coupled to the power generator, the power generator adapted to receive temperature information from the temperature sensor and adapted to adjust the power supplied to the ablation probe to prevent damage to the area of living tissue near the targeted area of living tissue.

2. The ablation system of claim 1, wherein the ablation probe introducer includes an ablation probe introducer hub.

3. The ablation system of claim 1, wherein the targeted area of living tissue is a tumor.

4. The ablation system of claim 1, wherein the power generator is an electrosurgical generator.

5. The ablation system of claim 4, wherein the electrosurgical generator automatically adjusts the power supplied to the ablation probe depending on the temperature information from the temperature sensor.

6. The ablation system of claim 4, wherein the electrosurgical generator adjusts the power supplied to the ablation probe by user input.

7. The ablation system of claim 1, further comprising:
a thermocouple probe that is separate from the ablation probe introducer.

8. The ablation system of claim 7, further comprising:
a thermocouple probe introducer including an outer surface and an inner surface, a proximal end having a proximal wall, and an opposite distal end having a distal wall, the distal wall of the thermocouple probe introducer having a distal aperture, the thermocouple probe introducer having a central longitudinal axis of the thermocouple probe introducer, and the thermocouple probe positioned within the thermocouple probe introducer.

9. The ablation system of claim 1, wherein the temperature sensor is positioned at the distal end of the ablation probe.

10. The ablation system of claim 1, further comprising;
a cooling unit connected to the ablation probe for providing cooling fluid to the ablation probe.

11. A method of delivering energy by an ablation system to a targeted area of living tissue and for monitoring a temperature of a proximate area of living tissue near the targeted area of living tissue to prevent damage to the proximate area of living tissue, the method comprising:
utilizing an ablation probe introducer having an outer surface and an inner surface, a proximal end having a proximal wall, and an opposite distal end having a distal wall, the distal wall of the ablation probe introducer having a distal aperture, and the ablation probe introducer having a central longitudinal axis passing through the proximal wall and the distal wall of the ablation probe introducer;
inserting an ablation probe into the ablation probe introducer, the ablation probe including a plurality of ablation probe electrodes at a distal end of the ablation probe to deliver energy to the targeted area of living tissue;
utilizing a temperature sensor proximate to the ablation probe, the temperature sensor determining the temperature of the proximate area of living tissue;
utilizing a power generator that supplies power to the ablation probe and the temperature sensor, the ablation probe and the temperature sensor being electrically coupled to the power generator;
receiving, at the power generator, temperature information from the temperature sensor; and
adjusting, at the power generator, the power supplied to the ablation probe to prevent damage to the area of living tissue near the targeted area of living tissue.

12. The method of claim 11, further comprising:
connecting an ablation probe introducer hub to the ablation probe introducer.

13. The method of claim 11, further comprising:
inserting the ablation probe into a tumor to deliver energy to the tumor.

14. The method of claim 11, wherein the utilizing of the power generator is utilizing an electrosurgical generator that supplies the power to the ablation probe and the temperature sensor.

15. The method of claim 11, wherein the adjusting of the power supplied to the ablation probe is automatically adjusted depending on the temperature information received from the temperature sensor.

16. The method of claim 11, wherein the adjusting of the power supplied to the ablation probe is adjusted by user input.

17. The method of claim 11, further comprising:
utilizing a thermocouple probe electronically coupled to the power generator, the thermocouple probe providing additional temperature information to the power generator.

18. The method of claim 17, further comprising:
inserting the thermocouple probe into the ablation probe introducer.

19. The method of claim 17, further comprising:
utilizing a thermocouple probe introducer including an outer surface and an inner surface, a proximal end having a proximal wall, and an opposite distal end having a distal wall, the distal wall of the thermocouple probe introducer having a distal aperture, the thermocouple probe introducer having a central longitudinal axis of the thermocouple probe introducer, and the thermocouple probe positioned within the thermocouple probe introducer.

20. The method of claim 11, further comprising:
utilizing a cooling unit connected to the ablation probe for providing cooling fluid to the ablation probe.
